(19) United States
(12) Patent Application Publication
(10) Pub. No.: US 2006/0052364 A1
(43) Pub. Date: Mar. 9, 2006

(54) Pharmaceutical composition for treating pain comprising oxicarbazepine, or derivatives thereof, and cox2 inhibitors

(76) Inventors: Margaret Hopwood, PARSIPPANY, NJ (US); Donald Manning, Bloomsbury, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/531,801

(22) PCT Filed: Oct. 17, 2003
(86) PCT No.: PCT/EP03/11555

§ 371 Date: Apr. 18, 2005

(30) Foreign Application Priority Data
Oct. 17, 2002 (GB) 0224199.0
Oct. 17, 2002 (GB) 0224200.6

(51) Int. Cl. A61K 31/55; A61K 31/365; A61K 31/18; A61K 31/415

(52) U.S. Cl. 514/217; 514/406; 514/471; 514/602

(57) ABSTRACT

A pharmaceutical composition for treatment of pain, comprises in combination oxcarbazepine or derivative thereof as defined and a COX-2 inhibitor for simultaneous, sequential or separate use. Also provided is a method of treating a patient suffering from pain, comprising administering to the patient an effective amount of oxcarbazepine or derivative thereof and an effective amount of a COX-2 inhibitor.

This invention relates to COX-2 inhibitors, in particular to combined use of COX-2 inhibitors with anti-epileptic compounds, and compositions containing such combinations.

Accordingly the invention provides a pharmaceutical composition for treatment of pain, which comprises in combination oxcarbazepine or derivative thereof of formula I 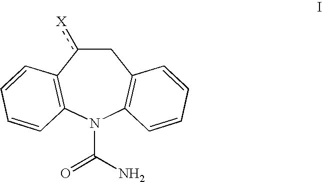
wherein X is ═O or —OH, the bond between the azepine ring and X being a double bond when X is ═O and a single bond when X is —OH, and a COX-2 inhibitor for simultaneous, sequential or separate use.

Further the invention provides the use of a COX-2 inhibitor for the preparation of a medicament, for use in combination with oxcarbazepine or derivative thereof of formula I as defined above, for treatment of pain.

In the alternative the invention provides use of oxcarbazepine or derivative thereof of formula I as defined above, for the preparation of a medicament for use in combination with a COX-2 inhibitor for treatment of pain.

In a further aspect the invention provides a method of treating a patient suffering from pain comprising administering to the patient an effective amount of oxcarbazepine or derivative thereof of formula I as defined above, and an effective amount of a COX-2 inhibitor.

In yet further aspects the invention provides:
(i) A package comprising oxcarbazepine or derivative thereof of formula I as defined above, together with instructions for use in combination with a COX-2 inhibitor for treatment of pain, or
(ii) A package comprising a COX-2 inhibitor together with instructions for use in combination with oxcarbazepine or derivative thereof of formula I as defined above, for treatment of pain.

Pain in general may be treated in accordance with the present invention including both nociceptive and inflammatory pain. In particular the combination treatment of the invention may be used for the treastment of musculoskeletal pain, especially lower back pain.

In the present description the terms “treatment” or “treat” refer to both prophylactic or preventative treatment as well as curative or disease modifying treatment, including treatment of patients at risk of suffering pain as well as patients who are already suffering pain.

The COX-2 inhibitors used in the pharmaceutical compositions and treatment methods of the present invention are typically those which have an IC₅₀ for COX-2 inhibition less than about 2 μM and an IC₅₀ for COX-1 inhibition greater than about 5 μM, e.g. when measured in the assays described by Brideau et al. in Inflamm. Res. 45:68-74 (1996). Preferably the COX-2 inhibitor has a selectivity ratio of at least 10, more preferably at least 40, for COX-2 inhibition over COX-1 inhibition.

Thus, for example, suitable COX-2 inhibitors for use in the invention may include the following compounds or derivatives thereof or a pharmaceutically acceptable salt thereof, or any hydrate thereof: rofecoxib, etoricoxib, celecoxib, valdecoxib, parecoxib, or a 5-alkyl-2-arylaminophenylacetic acid derivative COX-2 inhibitor, e.g. of formula V as defined below.

Alternative classes of COX-2 inhibitor compounds for use in the invention include those described in U.S. Pat. No. 6,136,804 (Merck).

COX-2 inhibitors of formula V are particularly preferred for use in the present invention.

Thus in preferred embodiments the COX-2 inhibitor for use in the present invention comprises a compound of formula V 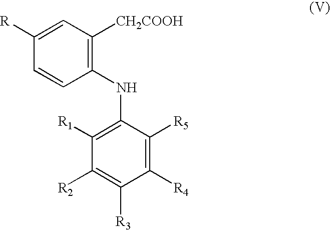
wherein R is methyl or ethyl;
R₁ is chloro or fluoro;
R₂ is hydrogen or fluoro;
R₃ is hydrogen, fluoro, chloro, methyl, ethyl, methoxy, ethoxy or hydroxy;
R₄ is hydrogen or fluoro; and
R₅ is chloro, fluoro, trifluoromethyl or methyl.

Above and elsewhere in the present description the terms “an oxcarbazepine or derivative thereof” and “COX-2 inhibitor” include, as appropriate, pharmaceutically acceptable salts and esters thereof.

Particularly preferred compounds of formula V are those wherein R is methyl or ethyl; R₁ is chloro or fluoro; R₂ is hydrogen; R₃ is hydrogen, fluoro, chloro, methyl or hydroxy; R₄ is hydrogen; and R₅ is chloro, fluoro or methyl; pharmaceutically acceptable salts thereof; and pharmaceutically acceptable esters thereof.

A particularly preferred embodiment relates to the compounds of formula V wherein R is methyl or ethyl; R₁ is fluoro; R₂ is hydrogen; R₃ is hydrogen, fluoro or hydroxy; R₄ is hydrogen; and R₅ is chloro; pharmaceutically acceptable salts thereof; and pharmaceutically acceptable prodrug esters thereof.

Another particularly preferred embodiment of the invention relates to compounds of formula V wherein R is ethyl or methyl; R₁ is fluoro; R₂ is hydrogen or fluoro; R₃ is hydrogen, fluoro, ethoxy or hydroxy; R₄ is hydrogen or fluoro; and R₅ is chloro, fluoro or methyl; pharmaceutically acceptable salts thereof; and pharmaceutically acceptable prodrug esters thereof.

Further are said compounds wherein R is methyl or ethyl; R₁ is fluoro; R₂-R₄ are hydrogen or fluoro; and R₅ is chloro or fluoro; pharmaceutically acceptable salts thereof; and pharmaceutically acceptable prodrug esters thereof.

A further embodiment of the invention relates to the compounds of formula V wherein R is methyl or ethyl; R₁ is fluoro; R₂ is fluoro; R₃ is hydrogen, ethoxy or hydroxy; R₄ is fluoro; and R₅ is fluoro; pharmaceutically acceptable salts thereof; and pharmaceutically acceptable prodrug esters thereof.

Another embodiment of the invention relates to the compounds of formula V wherein R is methyl; R₁ is fluoro; R₂ is hydrogen; R₃ is hydrogen or fluoro; R₄ is hydrogen; and R₅ is chloro; pharmaceutically acceptable salts thereof; and pharmaceutically acceptable prodrug esters thereof.

Particularly preferred embodiments of the invention relate to compounds of formula V
(a) wherein R is methyl; R₁ is fluoro; R₂ is hydrogen; R₃ is hydrogen; R is hydrogen; and R₅ is chloro; pharmaceutically acceptable salts thereof; and pharmaceutically acceptable prodrug esters thereof;
(b) wherein R is methyl; R₁ is fluoro; R₂ is hydrogen; R₃ is fluoro; R₄ is hydrogen; and R₅ is chloro; pharmaceutically acceptable salts thereof; and pharmaceutically acceptable prodrug esters thereof;
(c) wherein R is ethyl; R₁ is fluoro; R₂ is fluoro; R₃ is hydrogen; R₄ is fluoro; and R₅ is fluoro; pharmaceutically acceptable salts thereof; and pharmaceutically acceptable prodrug esters thereof; and
(d) wherein R is ethyl; R₁ is chloro; R₂ is hydrogen; R₃ is chloro; R₄ is hydrogen; and R₅ is methyl; pharmaceutically acceptable salts thereof; and pharmaceutically acceptable prodrug esters thereof.

Most preferably the COX-2 inhibitor of formula V is 5-methyl-2-(2′-chloro-6′-fluoroanilino)phenylacetic acid, or a salt or ester thereof.

Pharmaceutically acceptable prodrug esters of the compounds of formula V are ester derivatives which are convertible by solvolysis or under physiological conditions to the free carboxylic acids of formula V. Such esters are e.g. lower alkyl esters (such as the methyl or ethyl ester), carboxy-lower alkyl esters such as the carboxymethyl ester, nitrooxy-lower alkyl esters (such as the 4-nitrooxybutyl ester), and the like. Preferred prodrugs are the compounds of formula Ia 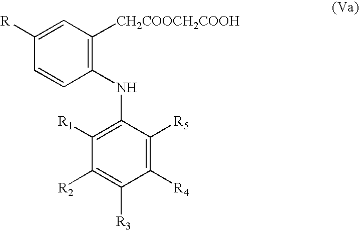
wherein R and R₁-R₅ have meaning as defined hereinabove for compounds of formula V; and pharmaceutically acceptable salts thereof.

The invention further provides a pharmaceutical composition for treatment of pain, which comprises in combination a carbamazepine or derivative thereof of formula II 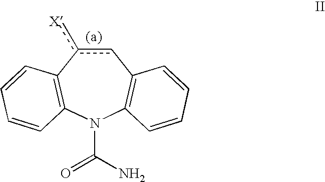
wherein X is ═O, —OH or H, the bond between the azepine ring and X′ being a double bond when X′ is ═O and a single bond when X′ is —OH or H and the bond (a) of the azepine ring being a single bond when X′ is ═O or —OH and a double bond when X′ is H, and a COX-2 inhibitor of formula VI 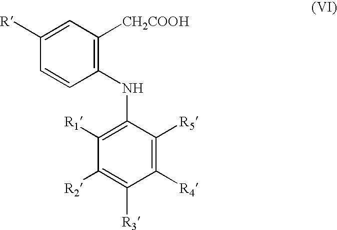
wherein R′ is methyl or ethyl;
R₁′ is chloro or fluoro;
R₂′ is hydrogen or fluoro;
R₃′ is hydrogen, fluoro, chloro, methyl, ethyl, methoxy, ethoxy or hydroxy;
R₄′ is hydrogen or fluoro; and
R₅′ is chloro, fluoro, trifluoromethyl or methyl; for simultaneous, sequential or separate use.

Further the invention provides the use of a COX-2 inhibitor of formula VI as defined above for the preparation of a medicament, for use in combination with a carbamazepine or derivative thereof of formula II as defined above for treatment of pain.

In the alternative the invention provides use of a carbamazepine or derivative thereof of formula II as defined above for the preparation of a medicament for use in combination with a COX-2 inhibitor of formula VI as defined above for treatment of pain.

In a further aspect the invention provides a method of treating a patient suffering from pain comprising administering to the patient an effective amount of a carbamazepine or derivative thereof of formula II as defined above and an effective amount of a COX-2 inhibitor of formula VI as defined above.

In yet further aspects the invention provides:
(i) A package comprising a carbamazepine or derivative thereof of formula II as defined above together with instructions for use in combination with a COX-2 inhibitor of formula VI as defined above for treatment of pain, or
(ii) A package comprising a COX-2 inhibitor of formula VI as defined above together with instructions for use in combination with a carbamazepine or derivative thereof of formula II as defined above for treatment of pain.

Particularly preferred compounds of formula VI are those wherein R′ is methyl or ethyl; R₁′ is chloro or fluoro; R₂′ is hydrogen; R₃′ is hydrogen, fluoro, chloro, methyl or hydroxy; R₄′ is hydrogen; and R₅′ is chloro, fluoro or methyl; pharmaceutically acceptable salts thereof; and pharmaceutically acceptable esters thereof.

A particularly preferred embodiment relates to the compounds of formula VI wherein R′ is methyl or ethyl; R₁′ is fluoro; R₂′ is hydrogen; R₃′ is hydrogen, fluoro or hydroxy; R₄′ is hydrogen; and R₅′ is chloro; pharmaceutically acceptable salts thereof; and pharmaceutically acceptable prodrug esters thereof.

Another particularly preferred embodiment of the invention relates to compounds of formula VI wherein R′ is ethyl or methyl; R₁′ is fluoro; R₂′ is hydrogen or fluoro; R₃′ is hydrogen, fluoro, ethoxy or hydroxy; R₄′ is hydrogen or fluoro; and R₅′ is chloro, fluoro or methyl; pharmaceutically acceptable salts thereof; and pharmaceutically acceptable prodrug esters thereof.

Further preferred are said compounds wherein R′ is methyl or ethyl; R₁′ is fluoro; R₂′-R₄′ are hydrogen or fluoro; and R₅′ is chloro or fluoro; pharmaceutically acceptable salts thereof; and pharmaceutically acceptable prodrug esters thereof.

A further embodiment of the invention relates to the compounds of formula VI wherein R′ is methyl or ethyl; R₁′ is fluoro; R₂′ is fluoro; R₃′ is hydrogen, ethoxy or hydroxy; R₄′ is fluoro; and R₅′ is fluoro; pharmaceutically acceptable salts thereof; and pharmaceutically acceptable prodrug esters thereof.

Another embodiment of the invention relates to the compounds of formula VI wherein R′ is methyl; R₁′ is fluoro; R₂′ is hydrogen; R₃′ is hydrogen or fluoro; R₄′ is hydrogen; and R₅′ is chloro; pharmaceutically acceptable salts thereof; and pharmaceutically acceptable prodrug esters thereof.

Particularly preferred embodiments of the invention relate to compounds of formula VI
(a) wherein R′ is methyl; R₁′ is fluoro; R₂′ is hydrogen; R₃′ is hydrogen; R₄′ is hydrogen; and R₅′ is chloro; pharmaceutically acceptable salts thereof; and pharmaceutically acceptable prodrug esters thereof;
(b) wherein R′ is methyl; R₁′ is fluoro; R₂′ is hydrogen; R₃′ is fluoro; R₄′ is hydrogen; and R₅′ is chloro; pharmaceutically acceptable salts thereof; and pharmaceutically acceptable prodrug esters thereof;
(c) wherein R′ is ethyl; R₁′ is fluoro; R₂′ is fluoro; R₃′ is hydrogen; R₄′ is fluoro; and R₅′ is fluoro; pharmaceutically acceptable salts thereof; and pharmaceutically acceptable prodrug esters thereof; and
(d) wherein R′ is ethyl; R₁′ is chloro; R₂′ is hydrogen; R₃′ is chloro; R₄′ is hydrogen; and R₅′ is methyl; pharmaceutically acceptable salts thereof; and pharmaceutically acceptable prodrug esters thereof.

Most preferably the COX-2 inhibitor of formula VI is 5-methyl-2-(2′-chloro-6′-fluoroanilino)phenylacetic acid, or a salt or ester thereof.

Pharmaceutically acceptable prodrug esters of the compounds of formula VI are ester derivatives which are convertible by solvolysis or under physiological conditions to the free carboxylic acids of formula VI. Such esters are e.g. lower alkyl esters (such as the methyl or ethyl ester), carboxy-lower alkyl esters such as the carboxymethyl ester, nitrooxy-lower alkyl esters (such as the 4-nitrooxybutyl ester), and the like. Preferred prodrugs are the compounds of formula VIa 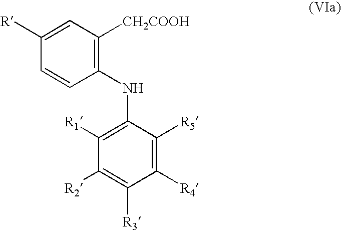
wherein R′ and R₁′-R₅′ have meaning as defined hereinabove for compounds of formula VI; and pharmaceutically acceptable salts thereof.

Carbamazepine is alternatively known as 5H-dibenz(b,f)azepine-5-carboxamide, G-32883, Biston, Calepsin, Carbelan, Epitol, Finlepsin, Sirtal, Stazepine, Tegretal, Tegretol, Telesmin and Timonil. The carbamazepine derivatives for use in the invention are alternatively known oxcarbazepine, GP-47680 and 10-oxo-10, 11-dihydro-5H-dibenz(b,f)azepine-5-carboxamide (Trileptal®) and 10-hydroxy-10,11-dihydro-5H-dibenz(b,f)azepine-5-carboxamide, MHD or GP47779 respectively.

Compounds of formula V, VI, Va and VIa and their synthesis are described in published international patent applications Nos. WO 99/11605 and WO 01/23346, the teachings of which are incorporated herein by reference.

Monohydroxycarbamazepine (10-hydroxy-10,11-dihydro-carbamazepine), the main metabolite of the antiepileptic oxcarbazepine (Trileptal™) is well known from the literature [see for example Schuetz H. et al., Xenobiotica (GB), 16(8), 769-778 (1986)] and can be prepared synthetically starting from oxcarbazepine according to conventional methods. Monohydroxycarbamazepine has been first disclosed in GB 1310120. The compound is indicated to be suitable for the treatment of psychosomatic disturbances, epilepsy, trigeminal neuralgia and cerebral spasticity.

Pharmacologically acceptable salts of oxcarbazepine derivative thereof and COX-2 inhibitors are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

The Agents of the Invention, i.e. the COX-2 inhibitor and the oxcarbazepine or derivative thereof are preferably used in the form of pharmaceutical preparations that contain the relevant therapeutically effective amount of of each active ingredient (either separately or in combination) optionally together with or in admixture with inorganic or organic, solid or liquid, pharmaceutically acceptable carriers which are suitable for administration. The Agents of the Invention may be present in the same pharmaceutical compositions, though are preferably in separate pharmaceutical compositions. Thus the active ingredients may be administered at the same time (e.g. simultaneously) or at different times (e.g. sequentially) and over different periods of time, which may be separate from one another or overlapping.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

The particular mode of administration and the dosage may be selected by the attending physician taking into account the particulars of the patient, especially age, weight, life style, activity level, and disease state as appropriate

Preferably, both the COX-2 inhibitor and oxcarbazepine or derivative pharmaceutical compositions are adapted for oral or parenteral (especially oral) administration. Intravenous and oral, first and foremost oral, adminstration is considered to be of particular importance. Preferably the COX-2 inhibitor active ingredient is in oral form.

The dosage of COX-2 inhibitor administered is dependent on the species of warm-blooded animal (mammal), the body weight, age and individual condition, and on the form of administration. A unit dosage for oral administration to a mammal of about 50 to 70 kg may contain between about 5 and 1500 mg, e.g. from 100-1000 mg, preferably 200-800 mg of the active ingredient.

COX-2 inhibitor formulations in single dose unit form contain preferably from about 1% to about 90%, and formulations not in single dose unit form contain preferably from about 0.1% to about 20%, of the active ingredient. Single dose unit forms such as capsules, tablets or dragées contain e.g. from about 1 mg to about 1500 mg of the active ingredient.

COX-2 inhibitor formulations in single dose unit form contain preferably from about 1% to about 90%, and formulations not in single dose unit form contain preferably from about 0.1% to about 20%, of the active ingredient. Single dose unit forms such as capsules, tablets or dragées contain e.g. from about 1 mg to about 1500 mg of the active ingredient.

Similarly the dosage of oxcabazepine or derivative administered is dependent on the species of warm-blooded animal (mammal), the body weight, age and individual condition, and on the form of administration. In general, the daily dosage of oxcarbazepine or derivative varies between about 3 mg/kg and about 20 mg/kg. Suitable unit dosage forms, such as dragées, tablets or suppositories, preferably contain 30-200 mg of oxcarbazepine or derivative. Dosage units for oral administration preferably contain between 10% and 90% by weight of oxcarbazepine or derivative.

Pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

The following examples are intended to illustrate the invention and are not to be construed as being limitations thereon.

### EXAMPLES

### A. Formulation Examples

### Example 1

TABLE 1

| | Amount per 200 mg |
|---|---|
| Ingredient | tablet batch (kg) |
| Core | |
| Granulation | |
| 5-methyl-2-(2′-chloro-6′- | 50** |
| fluoroanilino)phenylacetic acid drug | |
| substance | |
| Microcrystalline cellulose, NF (PH | 12.85 |
| 101) | |
| Lactose monohydrate, NF | 11.65 |
| Croscarmellose sodium, NF | 1 |
| Povidone, USP | 4 |
| Titanium dioxide, USP | 2 |
| Water, purified***, USP | 20.375 |
| Extra-granular Phase | |
| Microcrystalline cellulose, NF (PH | 13 |
| 102) | |
| Croscarmellose sodium, NF | 3 |
| Titanium dioxide, USP | 2 |
| Magnesium stearate, NF | 0.5 |
| Coating | |
| Opadry white | 2.801**** |
| Opadry yellow | 2.0**** |
| Opadry red | 0.4**** |
| Opadry black | 0.0504**** |
| Water, purified***, USP | 29.758**** |

**The weight of drug substance is taken with reference to the dried substance (100 percent) on the basis of the assay value (factorization). The difference in weight is adjusted by the amount of microcrystalline cellulose used.
***Removed during processing.
****Includes a 50% excess for loss during the coating process.

Table 1, above, sets out the formula for a batch of approximately 250,000 immediate release film-coated tablets of 5-methyl-2-(2′-chloro-6′-fluoroanilino)-phenylacetic acid. To make the tablets, titanium dioxide is dispersed in water, followed by the addition of povidone and mixing for 20 minutes to make a povidone/titanium dioxide suspension. The drug substance, lactose, microcrystalline cellulose, and croscarmellose are mixed in a high shear mixer (e.g., a Collette Gral) for 5 minutes to form a drug mixture. The drug mixture is granulated in the high shear mixer with the povidone/titanium dioxide suspension. The suspension is pumped at a rate of 3 kg/min into the drug mixture. The resulting mixture is mixed an additional 90 seconds after all the suspension is added. The wet granulation is dried in a fluid bed dryer, using an inlet air temperature of 50° C. The residual water target is 3.5% (with a permissible range of 2.5-4.5%). The dried granulation is passed through a screen using a mill (oscillator) and a 30 mesh screen. The previous steps are repeated to make a second granulation.

The extra-granular phase titanium dioxide is passed through a 60 mesh hand screen. The dry granulations are mixed with the extra-granular phase microcrystalline cellulose, croscarmellose sodium and titanium dioxide in a twin shell mixer for 300 revolutions to form a penultimate mixture. Magnesium stearate is passed through a 60 mesh hand screen and is mixed with the penultimate mixture in a twin shell mixer for 50 revolutions to form a tableting mixture. The tableting mixture is pressed into tablets using a tablet press and oval punches.

The coating powders (Opadry) are mixed with purified water to make a 15% w/w coating suspension. The tablets are film coated with the coating suspension in a coating pan using 60° C. to 75° C. inlet air temperature.

Table 2 sets out the contents of a 200 mg 5-methyl-2-(2′-chloro-6′-fluoroanilino)phenylacetic acid film-coated tablet.

TABLE 2

| | Theoretical | |
|---|---|---|
| Ingredient | amount [mg] | Function |
| Core | | |
| 5-methyl-2-(2′-chloro-6′- | 200 | Active |
| fluoroanilino)phenylacetic acid | | substance |
| drug substance | | |
| Microcrystalline cellulose (PH | 51.4 | Filler |
| 101) | | |
| Lactose | 46.6 | Filler |
| Povidone | 16 | Binder |
| Titanium dioxide | 8 | Color |
| Croscarmellose sodium | 4 | Disintegrant |
| Water, purified* | Q.S. | Granulating |
| | | liquid |
| Extragranular phase | | |
| Microcrystalline cellulose (PH | 52 | Filler |
| 102) | | |
| Croscarmellose sodium | 12 | Disintegrant |
| Titanium dioxide | 8 | Color |
| Magnesium stearate | 2 | Lubricant |
| Core weight | 400 | |
| Coating | | |
| Opadry white (00F18296) | 7.4676 | Color |
| Opadry yellow (00F12951) | 5.3312 | Color |
| Opadry red (00F15613) | 1.0668 | Color |
| Opadry black (00F17713) | 0.1344 | Color |
| Water, purified* | Q.S. | Coating solvent |
| Total weight | 414 | |

*removed during processing

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1

### Example 2

An alternative formulation is as set out in Table 3, with information about as percentage w/w, mg/dose, and kg/50,000 tablet batch.

(a) TABLE 3
Alternative formulation composition

| % w/w | Ingredient | Mg/dose | Kg/batch |
|---|---|---|---|
| | Granulation | | |
| 65.04 | 5-methyl-2-(2′-chloro-6′-fluoroanilino) | 400.00 | 20.00 |
| | phenylacetic acid drug substance | | |
| 2.15 | Croscarmellose sodium, NF (Ac-Di-Sol) | 13.22 | 0.661 |
| 6.60 | Povidone K30, USP | 40.59 | 2.029 |
| 18.12 | Purified water, USP* | Qs | Qs |
| | Blending | | |
| 23.56 | Microcrystalline Cellulose, NF (Avicel | 144.90 | 6.066 |
| | PH 102) | | |
| 2.15 | Croscarmellose sodium, NF (Ac-Di-Sol) | 13.22 | 0.553 |
| 0.50 | Magnesium Stearate, NF (vegetable | 3.07 | 0.128 |
| | source) | | |
| | Film Coating | | |
| 84.46 | Opadry, Global White 00F18296 | 15.2028 | 0.296637 |
| 14.03 | Opadry, Global Red 00F15613 | 2.5254 | 0.049275 |
| 1.51 | Opadry, Global Black 00F17713 | 0.2718 | 0.005303 |
| | Purified Water, USP* | Qs | 1.990218 |
| | Film Coated Tablet Weight | 633.00 | |

*Does not appear in final product. Percentage of water added used for granulation based on the dry weight of drug substance and croscarmellose sodium.

The batch is granulated as described in Example 1. The granulation is dried to residual moisture (% LOD) of 1.79%. The formulation process is the same as for the development batches as described above, except for the additional step of coating with Opadry in a coating pan. The coating powders (Opadry) are mixed with purified water to make a 15% w/w coating suspension. The tablets are film coated with the coating suspension in a coating pan using 60° C. to 75° C. inlet air temperature. Based on friability data, a target force of 18 KN (16-20 KN range) is used to compress the remainder of the batch, resulting in acceptable friability (less than 0.5%) and the disintegration times of less than 5 mins. The ejection force is approximately 800 N throughout the compression run. This demonstrates that the blend is lubricated adequately. No picking/sticking is observed on the punch surfaces after 225 minutes. Thus, a smaller size tablet with high drug loading (65%) is achieved using a high shear granulation process, using 17×6.7 mm ovaloid tooling to get tablets with acceptable hardness and friability characteristics.

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1%.

### Example 3

Wet granulated tablet composition

| Amount per tablet | Ingredient |
|---|---|
| 25 mg | COX-2 inhibitor |
| 79.7 mg | Microcrystalline cellulose |
| 79.7 mg | Lactose monohydrate |
| 6 mg | Hydroxypropyl cellulose |
| 8 mg | Croscarmellose sodium |
| 0.6 mg | Iron oxide |
| 1 mg | Magnesium stearate |

Tablet dose strengths of between 5 and 125 mg can be accomodated by varying total weight, and the ratio of the first three ingredients. Generally it is preferable to maintain a 1:1 ratio for microcrystalline cellulose: lactose monohydrate.

### Example 4

Hard gelatine capsule composition

| Amount per | | |
|---|---|---|
| capsule | | Ingredient |
| 25 | mg | COX-2 inhibitor |
| 37 | mg | Microcrystalline cellulose |
| 37 | mg | Lactose anhydrate |
| 1 | mg | Magnesium stearate |
| 1 | capsule | Hard gelatin capsule |

Capsule does strengths of between 1 and 50 mg can be accomodated by varying total fill weight, and the ratio of the first three ingredients. Generally it is preferable to maintain a 1:1 ratio for microcrystalline cellulose:lactose monohydrate.

### Example 5

Oral solution

| Amount per 5 mL | | Ingredient |
|---|---|---|
| 50 | mg | COX-2 inhibitor |
| to 5 | mL | with Polyethylene oxide 400 |

### Example 6

Intravenous infusion

| Amount per | | |
|---|---|---|
| 200 mL dose | | Ingredient |
| 1 | mg | COX-2 inhibitor |
| 0.2 | mg | Polyethylene oxide 400 |
| 1.8 | mg | Sodium chloride |
| to 200 | mL | Purified water |

### Example 7

TABLE 4
Oxcarbazepine Formulations

| | [mg] | [mg] |
|---|---|---|
| Tablet core | | |
| TRILEPTAL AS (oxcarbazepine) | 300.0 | 600.0 |
| extra fine | | |
| Cellulose HPM 603 | 8.4 | 16.8 |
| Microcrystalline Cellulose | 65.6 | 131.2 |
| Colloidal anhydrous silica | 1.6 | 3.2 |
| Magnesium stearate | 4.4 | 8.8 |
| Crosspovidone | 20.0 | 40.0 |
| Core Weight | 400.0 | 800.0 |
| Coating | | |
| Cellulose HPM 603 | 7.351 | 11.946 |
| Iron(II) oxide (yellow) 17268 | 0.499 | 0.811 |
| Polyethylene glycol (PEG) 8000 | 1.331 | 2.162 |
| Talcum | 5.323 | 8.649 |
| Titanium dioxide | 1.497 | 2.432 |
| Coating Weight | 16.000 | 26.000 |

Oxcarbazepine, cellulose HPM 603 (binder) and a portion (approximately half) of the microcrystalline cellulose (binder, filler, disintegration-promoting excipient) are mixed in a mixer, preferably in a high-speed mixer, e.g. DIOSNA, LOEDIGE, FIELDER or GLATT. Water is added to the mixture and and the mixture kneaded, preferably in a high-speed mixer until an adequate consistency is achieved. Alternatively, the HPM 603 may be dissolved in the water, beforehand. The mixture is formed into wet granules using an ALEXANDER Reibschnitzler, QUADRO-COMILL and the granules dried in a fluidised bed (AEROMATIC, GLATT). The remainder of the microcrystalline celluloseis added together with AEROSIL 200 (flow conditioner) and crospovidone (disintegrator) to the dry granules followed by mixing in a comminuter (FREWITT, QUADRO-COMILL, FITZMILL). Finally, magnesium stearate (lubricant) is added with mixing (STOECKLIN container mixer, VRIECO mixer). Alternatively, the lubricant may be added directly to the comminuted material. The final mixture is compressed to form TRILEPTAL tablets (eccentric press, rotary press: KILIAN, KORSCH, FETTE, MANESTY).

The tablets are coated with an aqueous preparation consisting of cellulose HPM 603 (film former), iron oxide yellow 17268 (pigment), PEG 8000 (plasticiser for the film former), talcum (anti-adhesive agent, covering agent) and titanium dioxide (covering agent) in a rotating coating pan (ACCELA-COTA, GLATT, DRIACOATER, DUMOULIN). Alternatively, it is possible to use, for example, fluidised-bed or air-suspension apparatus for the coating process (AEROMATIC, GLATT, FREUND, HUETTLIN).

### Example 8

An alternative formulation is as set out in Table 5, with information about as percentage w/w, mg/dose, and kg/50,000 tablet batch.

EXAMPLE 1

| | (mg) | (mg) | (mg) |
|---|---|---|---|
| Tablet Core: | | | |
| Oxcarbazepine | 150 | 300 | 600 |
| Avicel PH 102 | 32.8 | 65.6 | 131.2 |
| Cellulose HPM 603 | 4.2 | 8.4 | 16.8 |
| Polyvinylpyrrolidone | 10 | 20 | 40 |
| Aerosil 200 | 0.8 | 1.6 | 3.2 |
| Magnesium stearate | 2.2 | 4.4 | 8.8 |
| | 200 | 400 | 800 |
| Coating: | | | |
| Polyethylene glycol (PEG) | 0.832 | 1.331 | 2.162 |
| 8000 | | | |
| Cellulose HPM 603 | 4.595 | 7.352 | 11.947 |
| Talcum | 3.327 | 5.323 | 8.649 |
| Titanium Dioxide | 0.935 | 1.496 | 2.431 |
| Iron oxide, yellow | 0.312 | 0.499 | 0.81 |
| | 10 | 16 | 26 |
| Total | 210 | 416 | 826 |

Mix the TRILEPTAL, cellulose HPM 603 (binder) and AVICEL PH 102 (binder, filler, disintegration-promoting excipient) in a mixer, preferably in a high-speed mixer (DIOSNA, LOEDIGE, FIELDER, GLATT etc.). Add water as granulation liquid to the mixture, and knead in a mixer, preferably a high-speed mixer, until an adequate consistency is achieved. Alternatively, the binder cellulose HPM may be dissolved in the granulation liquid, water, beforehand. Granulate the wet granules using a suitable device (ALEXANDER Reibschnitzler, QUADRO-COMILL) and dry in a fluidised bed (AEROMATIC, GLATT). Add AVICEL PH 102, AEROSIL 200 (flow conditioner) and polyvinylpyrrolidone PXL (disintegrator) to the dry granules and comminute and mix in a comminuter (FREWITT, QUADRO-COMILL, FITZMILL). Finally, add magnesium stearate (lubricant) and mix (STOECKLIN container mixer, VRIECO mixer). Alternatively, the lubricant may be added directly to the comminuted material. Compress the final mixture to form TRILEPTAL tablets (eccentric press, rotary press: KILIAN, KORSCH, FETTE, MANESTY).

Coat the tablets with an aqueous preparation consisting of cellulose HPM 603 (film former), iron oxide yellow 17268 (pigment), PEG 8000 (plasticiser for the film former), talcum (anti-adhesive agent, covering agent) and titanium dioxide (covering agent) in a rotating coating pan (ACCELA-COTA, GLATT, DRIACOATER, DUMOULIN). Alternatively, it is possible to use, for example, fluidised-bed or air-suspension apparatus for the coating process (AEROMATIC, GLATT, FREUND, HUETTLIN).

### Example 9

A typical oral solution of oxcarpazepine comprises
(% expressed in mass/volume):

| | |
|---|---|
| Oxcarbazepine, micronized, | 1 to 20% (e.g. |
| | TRILEPTAL/AS, extra fine) |
| Avicel RC 591 | 0.1 to 1.9% |
| Methylparaben | 0.01 to 1% |
| Polyethylene glycol 400 monostearate | 0.01 to 1% |
| Propylene glycol (1,2-propanediol), dist. | 0.5 to 10% |
| Propylparaben | 0.005 to 0.5% |
| Saccharin sodium, cryst. | 0.005 to 0.5% |
| Sorbic acid | 0.005 to 0.5% |
| Sorbitol solution (non crystallizing) | 10 to 40% |
| Ascorbic acid | 0.1 to 10% |
| Water, purified | 40 to 85% |
| Yellow plum-lemon aroma | 0 to 15% |

### Example 10

Parenteral Formulation with oxcarbazepine or a derivative thereof of formula I: Oxcarbazepine or a derivative thereof is dissolved under a nitrogen blanket with stirring at 60-80° C. in water for injection (WFI) at a concentration of 2.5 mg/ml. After cooling to room temperature anhydrous glucose for injection is added and dissolved by stirring under nitrogen purging to obtain a 4.75% concentration glucose in water. After filtration through 0.22 micrometer pore size filter, the solution is purged with nitrogen, filled in glass vials (class II quality), sealed with a rubber closure and alu-cap and sterilized by autoclaving at 121° C. for 15 minutes.

The vials are stable and clear of coloured particles for at least two years at 2-8° C.

### Example 11

Alternative parenteral Formulation with oxcarbazepine or a derivative thereof of formula I: Oxcarbazepine or a derivative thereof dissolved with stirring at 60-80° C. in WFI at a concentration of 2.5 mg/ml. After cooling to room temperature glucose for injection (anhydrous) is added and dissolved by stirring to obtain a 4.75% concentration of glucose in water. After filtration through 0.22 micrometer pore size filter, the solution is filled in glass vials, sealed with a rubber closure and alu-cap and sterilized by autoclaving at 121° C. for 15 minutes.

The vials are stable and clear of coloured particles for at least three months at 2-8° C.

### Example 12

Alternative parenteral Formulation with oxcarbazepine or a derivative thereof of formula I: Oxcarbazepine or a derivative thereof is dissolved under a nitrogen blanket by stirring at 60-80° C. in WFI at a concentration of 2.5 mg/ml. After cooling to room temperature sodium chloride is added and dissolved with stirring under nitrogen purging to obtain a 0.9% concentration of sodium chloride in water. After filtration through 0.22 micrometer pore size filter, the solution is purged with nitrogen, filled in glass vials, sealed with a rubber closure and alu-cap and sterilized by autoclaving at 121° C. for 15 minutes. The vials are inspected after three months storage at 2-8° C. and show the presence of red coloured particles.

### Example 13

Alternative parenteral Formulation with oxcarbazepine or a derivative thereof of formula I: Oxcarbazepine or a derivative thereof is dissolved with stirring at 60-80° C. in WFI at a concentration of 2.5 mg/ml. After cooling to room temperature sodium chloride is added and dissolved by stirring to obtain a 0.9% concentration of sodium chloride in water. After filtration through 0.22 micrometer pore size filter, the solution is filled in glass vials, sealed with a rubber closure and alu-cap and sterilized by autoclaving at 121° C. for 15 minutes. The solutions in the vials show within six weeks storage at 2-8° C. the presence of red coloured particles.

### Example 14

Carbamazepine Formulations:

| Core | | |
|---|---|---|
| Carbamazepine - water free (Tegretol ®) | 200 | mg |
| Microcrystalline Cellulose (Avicel ® - FMC - | 20 | mg |
| Corporation Philadelphia) | | |
| Hydroxypropylmethyl cellulose (Pharmacoat ® 603 - | 12.5 | mg |
| Shin-Etsu Chem. Co. - Tokyo) | | |
| Vinylpyrrolidone/Vinylacetate-60:40-Copolymeres | 80 | mg |
| (Kollidon ® VA 64 - BASF Ludwigshafen) | | |
| Polyethylenglycol (MG: 5 × 10⁶ -Polyox ® - Coagulant - | 80 | mg |
| Union Carbide) | | |
| Sodium chloride (purest) | 80 | mg |
| Sodium laurylsulfate | 6 | mg |
| Magnesium stearate | 11.5 | mg |
| | = 490 | mg |
| Coating | | |
| Cellulose acetate (32.0) | 16 | mg |
| Cellulose acetate (39.8) | 20 | mg |
| Polyethylenglycol 4000 | 4 | mg |
| | = 40 | mg |
| Total weight | 530 | mg |

Alternative Formulation:

| Core | | |
|---|---|---|
| Carbamazepine - water free (Tegretol ®) | 200 | mg |
| Microcrystalline Cellulose (Avicel ® - FMC - | 20 | mg |
| Corporation Philadelphia) | | |
| Hydroxypropylmethyl cellulose (Pharmacoat ® 603 - | 13 | mg |
| Shin-Etsu Chem. Co. - Tokyo) | | |
| Vinylpyrrolidone/Vinylacetate-60:40-Copolymeres | 80 | mg |
| (Kollidon ® VA 64 - BASF Ludwigshafen) | | |
| Hydroxyethyl cellulose (Tylose H 4000 PHA) | 80 | mg |
| Glucose (purest) | 90 | mg |
| Sodium laurylsulfate | 7 | mg |
| Magnesium stearate | 10 | mg |
| | = 500 | mg |
| Coating | | |
| Cellulose acetate (32.0) | 16 | mg |
| Cellulose acetate (39.8) | 20 | mg |
| Polyethylenglycol 4000 | 4 | mg |
| | = 40 | mg |
| Total weight | 540 | mg |

Alternative Formulation:

| Core | | |
|---|---|---|
| Carbamazepine - water free (Tegretol ®) | 200 | mg |
| Microcrystalline Cellulose (Avicel ® - FMC - | 20 | mg |
| Corporation Philadelphia) | | |
| Hydroxypropylmethyl cellulose (Pharmacoat ® 603 - | 12.5 | mg |
| Shin-Etsu Chem. Co. - Tokyo) | | |
| Vinylpyrrolidone/Vinylacetate-60:40-Copolymeres | 81.3 | mg |
| (Kollidon ® VA 64 - BASF Ludwigshafen) | | |
| Polyethylenglycol (MG:5 × 10⁶ -Polyox ® - Coagulant - | 80 | mg |
| Union Carbide) | | |
| Sodium laurylsulfate | 6 | mg |
| Magnesium stearate | 10.2 | mg |
| | = 410 | mg |
| Coating | | |
| Cellulose acetate (32.0) | 16 | mg |
| Cellulose acetate (39.8) | 20 | mg |
| Polyethylenglycol 4000 | 4 | mg |
| | = 40 | mg |
| Total weight | 450 | mg |

Alternative Formulation:

| Core | | |
|---|---|---|
| Carbamazepine - water free (Tegretol ®) | 200 | mg |
| Hydroxypropylmethyl cellulose (Pharmacoat ® 603 - | 25 | mg |
| Shin-Etsu Chem. Co. - Tokyo) | | |
| Hydroxyethyl cellulose (Natrosol ® - 250L - Hercules) | 25 | mg |
| Hydroxyethyl cellulose (Natrosol ® - 250H) | 25 | mg |
| Mannit | 215 | mg |
| Sodium laurylsulfate | 5 | mg |
| Magnesium stearate | 5 | mg |
| | = 500 | mg |
| Coating | | |
| Cellulose acetate (32.0) | 18.9 | mg |
| Cellulose acetate (39.8) | 2.9 | mg |
| Hydroxypropylmethyl cellulose 15 CPS | 2.1 | mg |
| Polyethylenglycol 8000 | 2.1 | mg |
| | = 26 | mg |
| Total weight | 526 | mg |

### Example 14

### Treatment of Patients

Assumptions:

1) Two formulations: 200 mg Prexige plus 300 mg Trileptal
   200 mg Prexige Plus 600 mg Trileptal
2) b.i.d. dosing
3) limited titration
4) effective dose Trileptal=900-1200 mg/day effective dose Prexige=400 mg/day
5) sample size would have to estimated by a statistician
6) trial timeline is set up to achieve POC but possibly not statistical significance
   Design: Double-Blind, Placebo-Controlled, Parallel Group, Multicenter
   Duration: 4 to 6 Weeks Including Screening
   Patient Population:
   inclusion criteria—male or female≧to 18 years old
      low back pain (below T6 and above gluteal fold) that may radiate to leg
      pain lasting more than three months
      pain present on five out of seven days
      VAS score≧to 40 mm on four of the last seven days
      comprehensive history and physical examination including focused neurological examination
   exclusion criteria—unstable spinal segment
      progressive neurological deficits
      excluded drugs: all other NSAIDs, opioids, TCAs, AEDs, oral steroids except for treatment of asthma or skin conditions, steroid injections
      other pain conditions that may interfere with assessment of the low back pain
      patients previously treated with either Prexige or Trileptal
      patients with hypersensitivity to carbamazepine, oxcarabazepine or lumiracoxib and other non-steroidal anti-inflammatories including aspirin
      patients with active disability compensation claims or any litigation related to their radiculopathic pain.
         Variables:
   primary efficacy variable—VAS
   secondary efficacy variables—responder rate, sleep assessment, SF-36, POMS, assessment of back mobility and low-back pain specific QOL, alternatively the use of the short form McGill pain questionnaire.

Suggested Visit Schedule

| | |
|---|---|
| visit 1 (day - 14 to day - 1) | screening |
| visit 2 (day 1) | randomization, titration and treatment |
| visit 3 (day 21) | withdrawal |
| visit 4 (day 28) | final visit |

The withdraw phase can be eliminated to give 4 weeks total treatment (1 week titration, 3 weeks maintenance).

Titration and Maintenance Dosing Schedule:

| Day | AM Dose^{a} | PM Dose^{a} | Total Daily Doses^{a} |
|---|---|---|---|
| 1 | 0 | 200/300 | 200/300 |
| 2 | 200/300 | 200/300 | 400/600 |
| 3 | 200/300 | 200/300 | 400/600 |
| 4 | 200/300 | 200/600 | 400/900 |
| 5 | 200/300 | 200/600 | 400/900 |
| 6 | 200/600 | 200/600 | 400/1200 |
| 7-21 | 200/600 | 200/600 | 400/1200 |
| 22-28 | 0 | 0 | 0 |

^{a}expressed as mg Prexige/mg Trileptal

1. A pharmaceutical composition for treatment of pain, which comprises in combination oxcarbazepine or derivative thereof of formula I 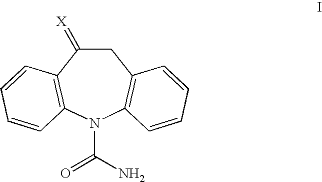
wherein X is ═O or —OH, the bond between the azepine ring and X being a double bond when X is ═O and a single bond when X is —OH, and a COX-2 inhibitor for simultaneous, sequential or separate use.

2. (canceled)

3. (canceled)

4. A method of treating a patient suffering from pain comprising administering to the patient an effective amount of oxcarbazepine or derivative thereof of formula I, 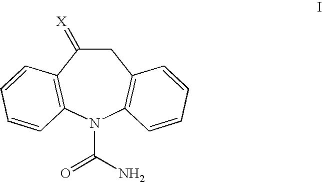
wherein X is ═O or —OH, the bond between the azepine ring and X being a double bond when X is ═O and a single bond when X is —OH, and an effective amount of a COX-2 inhibitor.

5. A package comprising oxcarbazepine or derivative thereof of formula I 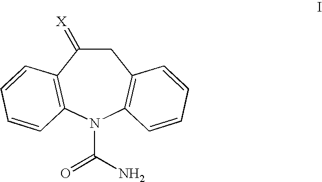
wherein X is ═O or —OH, the bond between the azepine ring and X being a double bond when X is ═O and a single bond when X is —OH, for treatment of pain according to a method wherein said oxcarbazepine or derivative thereof of formula I is used in combination with a COX-2 inhibitor or a package comprising a COX-2 inhibitor for treatment of pain according to a method wherein said COX-2 inhibitor is used together with instructions for use in combination with oxcarbazepine or derivative thereof of formula I as defined above.

6. A composition according to claim 1 in which the COX-2 inhibitor is selected from the group consisting of rofecoxib, etoricoxib, celecoxib, valdecoxib, parecoxib, and a 5-alkyl-2-arylaminophenylacetic acid derivative COX-2 inhibitor, or a pharmaceutically acceptable salt or any hydrate thereof.

7. A composition according to claim 1, in which the COX-2 inhibitor is a compound of formula V 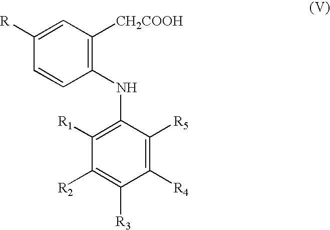
wherein R is methyl or ethyl;
R₁ is chloro or fluoro;
R₂ is hydrogen or fluoro;
R₃ is hydrogen, fluoro, chloro, methyl, ethyl, methoxy, ethoxy or hydroxy;
R₄ is hydrogen or fluoro; and
R₅ is chloro, fluoro, trifluoromethyl or methyl,
or a pharmaceutically acceptable salt or ester thereof.

8. A composition according to claim 7 in which the COX-2 inhibitor is 5-methyl-2-(2′-chloro-6′-fluoroanilino)phenylacetic acid,
or a pharmaceutically acceptable salt or ester thereof.

9. A pharmaceutical composition for treatment of pain, which comprises in combination oxcarbazepine or derivative thereof of formula II 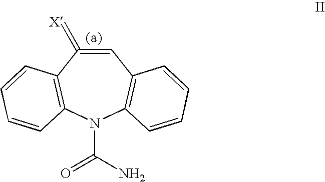
wherein X′ is ═O, —OH or H, the bond between the azepine ring and X′ being a double bond when X′ is ═O and a single bond when X′ is —OH or H and the bond (a) of the azepine ring being a single bond when X′ is ═O or —OH and a double bond when X′ is H, and a COX-2 inhibitor of formula VI 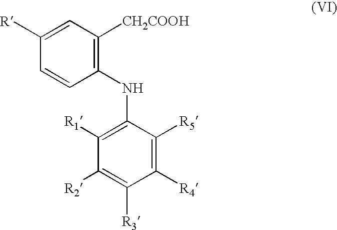
wherein R′ is methyl or ethyl;
R₁′ is chloro or fluoro;
R₂′ is hydrogen or fluoro;
R₃′ is hydrogen, fluoro, chloro, methyl, ethyl, methoxy, ethoxy or hydroxy;
R₄′ is hydrogen or fluoro; and
R₅′ is chloro, fluoro, trifluoromethyl or methyl;
for simultaneous, sequential or separate use.

10. (canceled)

11. (canceled)

12. A method of treating a patient suffering from pain comprising administering to the patient an effective amount of oxcarbazepine or derivative thereof of formula II, 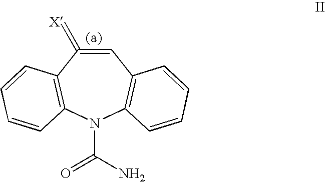
wherein X′ is ═O, —OH or H, the bond between the azepine ring and X′ being a double bond when X′ is ═O and a single bond when X′ is —OH or H and the bond (a) of the azepine ring being a single bond when X′ is ═O or —OH and a double bond when X′ is H, and an effective amount of a COX-2 inhibitor of formula VI 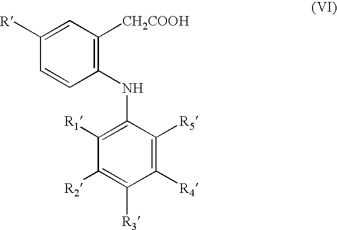
wherein R′ is methyl or ethyl;
R₁′ is chloro or fluoro;
R₂′ is hydrogen or fluoro;
R₃′ is hydrogen, fluoro, chloro, methyl, ethyl, methoxy, ethoxy or hydroxy;
R₄′ is hydrogen or fluoro; and
R₅′ is chloro, fluoro, trifluoromethyl or methyl.

13. A package comprising oxcarbazepine or derivative thereof of formula II 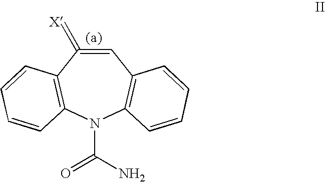
wherein X′ is ═O, —OH or H, the bond between the azepine ring and X′ being a double bond when X′ is ═O and a single bond when X′ is —OH or H and the bond (a) of the azepine ring being a single bond when X′ is ═O or —OH and a double bond when X′ is H, for treatment of pain according to a method wherein said oxcarbazepine or derivative thereof of formula II is used in combination with a COX-2 inhibitor of formula VI 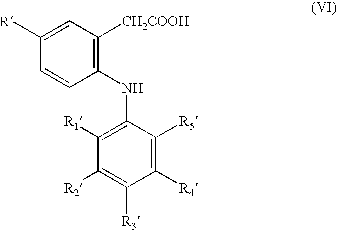
wherein R′ is methyl or ethyl;
R₁′ is chloro or fluoro;
R₂′ is hydrogen or fluoro;
R₃′ is hydrogen, fluoro, chloro, methyl, ethyl, methoxy, ethoxy or hydroxy;
R₄′ is hydrogen or fluoro; and
R₅′ is chloro, fluoro, trifluoromethyl or methyl, or a package comprising a COX-2 inhibitor of formula VI as defined above for treatment of pain according to a method wherein said COX-2 inhibitor is used in combination with oxcarbazepine or derivative thereof of formula II as defined above.

14. A composition according to claim 9 in which the COX-2 inhibitor is 5-methyl-2-(2′-chloro-6′-fluoroanilino)phenylacetic acid,
or a pharmaceutically acceptable salt or ester thereof.

15. A composition according to claim 9 in which the carbamazepine derivative is oxcarbazepine.

16. A composition according to claim 9 in which the carbamazepine derivative is 10-hydroxy-10, 11-dihydro-5H-dibenz(b,f)azepine-5-carboxamide.
